(19) United States
(12) Patent Application Publication
(10) Pub. No.: US 2014/0031725 A1
(43) Pub. Date: Jan. 30, 2014

(54) SYSTEM FOR ANALYZING THE WALKING PATTERN OF A USER

(71) Applicants: Jean Hong Jeon, Seoul (KR); FNT Co., Ltd., Cheonan-Si (KR)

(72) Inventor: Jean Hong Jeon, Seoul (KR)

(73) Assignees: FNT CO., LTD., Cheonan-Si (KR); Jean Hong Jeon, Seoul (KR)

(21) Appl. No.: 14/040,268

(22) Filed: Sep. 27, 2013

Related U.S. Application Data
(63) Continuation of application No. PCT/KR2012/002391, filed on Mar. 30, 2012.

(30) Foreign Application Priority Data
Mar. 31, 2011 (KR) 10-2011-0029365

(51) Int. Cl. A61B 5/11; A61B 5/00

(52) U.S. Cl. CPC A61B 5/112; A61B 5/6807; A61B 5/0004
USPC 600/595

(57) ABSTRACT

Disclosed is a system for analyzing the walking pattern of a user, comprising: a receiving unit which receives, from a sensor module installed in a moving object that moves in a pattern corresponding to a tracing of the movement of the feet of a user, rolling information detected in the advancing direction of the moving object; a pattern-analyzing unit, which analyzes the rolling information on the moving object received by the receiving unit to determine a pattern contacting the ground in the advancing direction of the moving object; and a walking pattern determining unit, which determines a walking pattern of the user based on the ground contacting pattern of the moving object determined by the pattern-analyzing unit.

### CROSS-REFERENCE TO RELATED APPLICATIONS

The present application is a continuation of International Application No. PCT/KR2012/002391 filed on Mar. 30, 2012, which claims priority to Korean Application No. KR 10-2011-0029365 filed on Mar. 31, 2011, which applications are incorporated herein by reference.

### TECHNICAL FIELD

The present invention relates to a system for analyzing the walking pattern of a pedestrian, which can detect the pattern of the gait of a pedestrian, that is, the walking pattern of the pedestrian.

### BACKGROUND ART

Generally, the walking pattern, that is, the gait, of a pedestrian means that a normal person dynamically moves the center of a body via the alternative motion of two legs, and is a unique pattern occurring while the rotational motion of each joint is changed to the translational motion of the joint.

Respective persons may have walking patterns having different characteristics, which may be mainly classified into three types: a normal walking pattern, that is, a so-called parallel-footed gait in which the two feet are moved in parallel along a movement direction based on footprints; a so-called out-toed gait in which the balls of the feet are outwardly spread compared to the parallel-footed gait; and an in-toed gait (a gait in which the balls of the feet are turned inward) which is contrary to the out-toed gait.

Such respective walking patterns are formed by persons' walking habits over a long time, and it is known that human bodies may be strained more or strained less depending on walking patterns and normal walking patterns are beneficial for health.

Recently, various types of methods for analyzing the walking patterns of respective pedestrians have been attempted and, as one of these methods, a method of installing a plurality of sensors on shoes, analyzing signals detected from the plurality of sensors, and then checking walking patterns has been presented.

However, since a plurality of sensors, that is, sensors for detecting yawing angles (gyro sensors) and a plurality of acceleration sensors for respectively detecting pitching angles and rolling angles, must be installed on shoes in the prior art, there is a problem in that the configuration of the shoes is complicated and cost is increased.

### SUMMARY

Accordingly, the present invention has been made keeping in mind the above problems, and an object of the present invention is to provide a system for analyzing the walking pattern of a pedestrian, which can detect the walking pattern of a pedestrian using a simple configuration.

A system for analyzing a walking pattern of a pedestrian according to the present invention to accomplish the above object includes a sensor module installed on a moving object moving in a pattern corresponding to a movement trajectory of a foot of a pedestrian and configured to detect rolling information based on a movement direction of the pedestrian as an axis; a reception unit configured to receive the rolling information detected by the sensor module; a pattern analysis unit configured to analyze the rolling information of the moving object received through the reception unit and determine a rolling direction of the moving object after the moving object touches a ground; and a walking pattern determination unit configured to determine a walking pattern of the pedestrian based on the rolling direction of the moving object after the moving object touches the ground, the rolling direction being determined by the pattern analysis unit.

In this case, the sensor module may include a 1-axis accelerometer for detecting an acceleration direction from a time point at which the moving object initially touches the ground to a time point at which the moving object fully touches the ground.

Further, the sensor module may include a 3-axis accelerometer for detecting an acceleration direction from a time point at which the moving object initially touches the ground to a time point at which the moving object fully touches the ground.

Furthermore, the pattern analysis unit may analyze whether the acceleration direction of the moving object detected by the sensor module is an inward direction or an outward direction of the pedestrian.

Furthermore, the walking pattern determination unit may be configured to, if the acceleration direction of the moving object is analyzed as being the inward direction of the pedestrian by the pattern analysis unit, determine that the walking pattern is that of an in-toed gait, if the acceleration direction of the moving object is analyzed as being the outward direction of the pedestrian, determine that the walking pattern is that of an out-toed gait, and if the acceleration direction is analyzed as being not biased to the inward or outward direction of the pedestrian, determine that the walking pattern is that of a normal gait.

In accordance with the system for analyzing the walking pattern of a pedestrian according to embodiments of the present invention, even in a state in which a minimum number of sensor modules, that is, a single sensor module, is installed in any one shoe, output data provided by the single sensor module is analyzed, and thus the walking pattern of the pedestrian may be accurately analyzed and determined.

In this way, there is an advantage in that the walking pattern of a pedestrian may be analyzed using the slightest simple configuration, thus enabling the walking pattern of the pedestrian to be analyzed with the least cost compared to the prior art.

Further, there is an advantage in that the output data of the sensor module may be collected via wireless communication with the sensor module and walking patterns may be analyzed, thus allowing anyone to easily and conveniently check and determine his or her walking pattern.

### BRIEF DESCRIPTION OF DRAWINGS

FIG. 1 is a schematic block diagram showing a system for analyzing the walking pattern of a pedestrian according to an embodiment of the present invention;

FIGS. 2A and 2B are schematic diagrams showing a state in which the sensor module of FIG. 1 is installed on a shoe;

FIG. 3 is a diagram showing the pitching operation and rolling operation of a shoe when a pedestrian walks, which shows an example of an out-toed gait;

FIG. 4 is a diagram showing the rolling operation of an in-toed gait;

FIG. 5 is a diagram showing a normal gait;

FIG. 6A is a diagram showing the walking pattern of an out-toed gait;

FIG. 6B is a diagram showing the walking pattern of an in-toed gait; and

FIG. 6C is a diagram showing the walking pattern of a normal gait.

### DETAILED DESCRIPTION

Hereinafter, a system for analyzing the walking pattern of a pedestrian according to embodiments of the present invention will be described in detail with reference to the attached drawings.

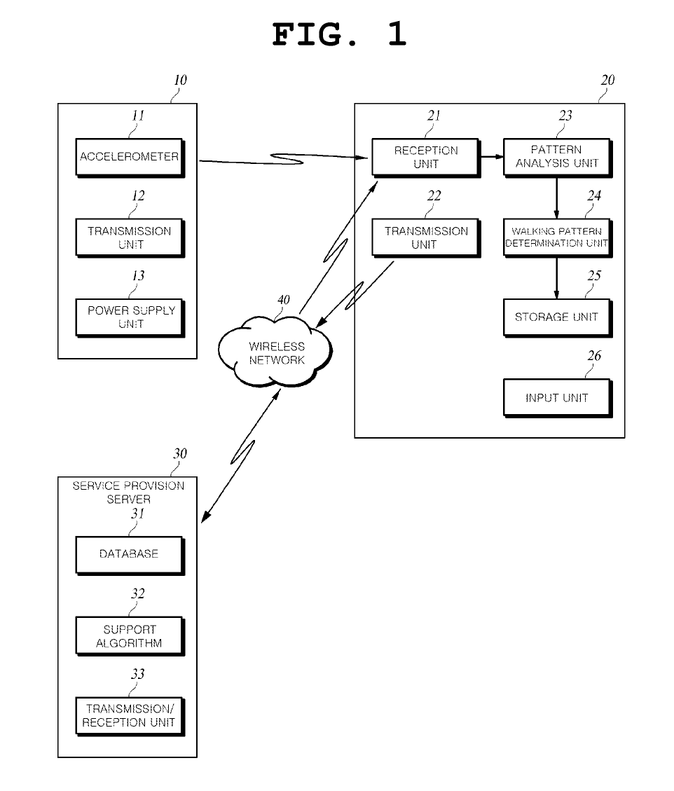

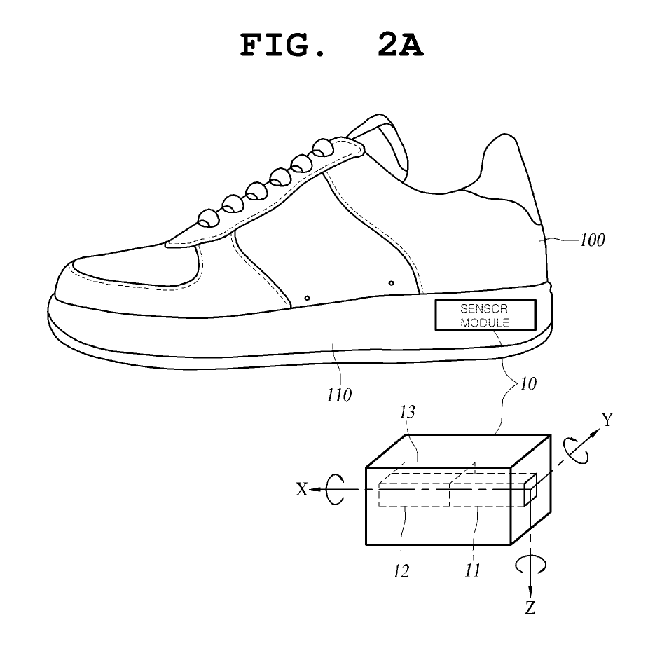

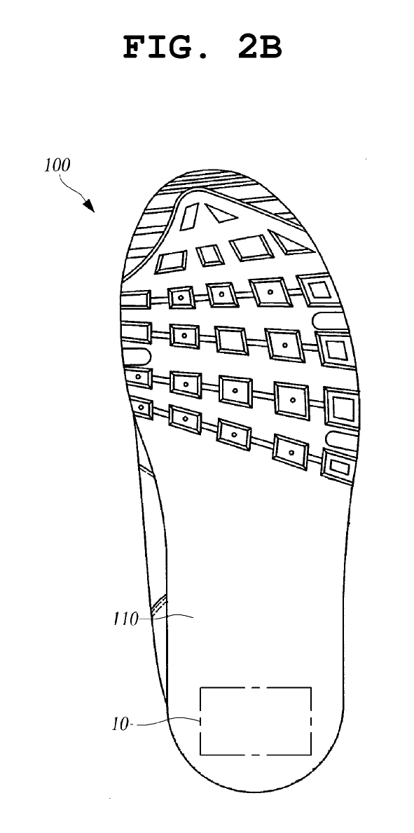

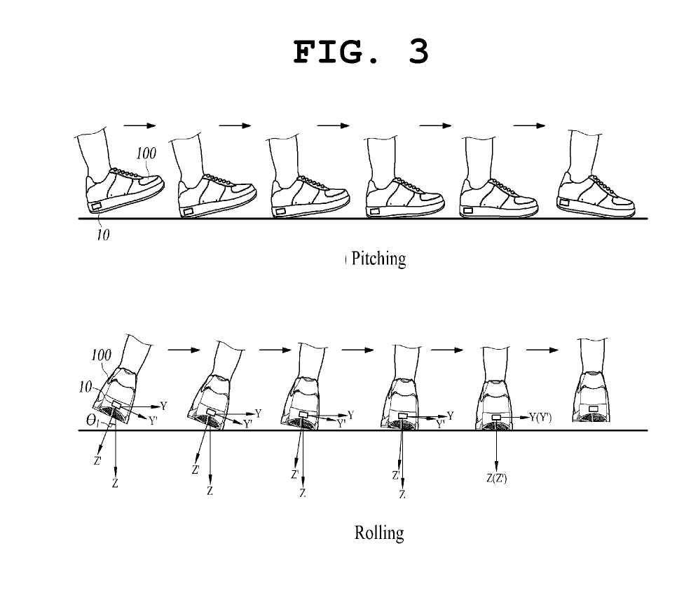

Referring to FIG. 1, FIGS. 2A and 2B, and FIG. 3, a system 20 for analyzing the walking pattern of a pedestrian according to an embodiment of the present invention includes a reception unit 21 for receiving rolling information detected along the movement direction x of a moving object 100 (hereinafter referred to as a “shoe”) moving in a pattern corresponding to the movement trajectory of the foot of the pedestrian from a sensor module 10 installed on the shoe 100, a transmission unit 22, a pattern analysis unit 23, a walking pattern determination unit 24, a storage unit 25, and an input unit 26.

The sensor module 10 may be installed to be attached to the outsole 110 of the shoe 100 or to be embedded in the internal portion of the shoe 100 and may preferably be installed on the heel of the shoe 100 based on the longitudinal length of the shoe 100 and in the center portion of the shoe 100 based on the lateral width of the shoe 100. Such a sensor module 10 may be installed on one shoe 100, that is, one of the left and right shoes, or on both the shoes.

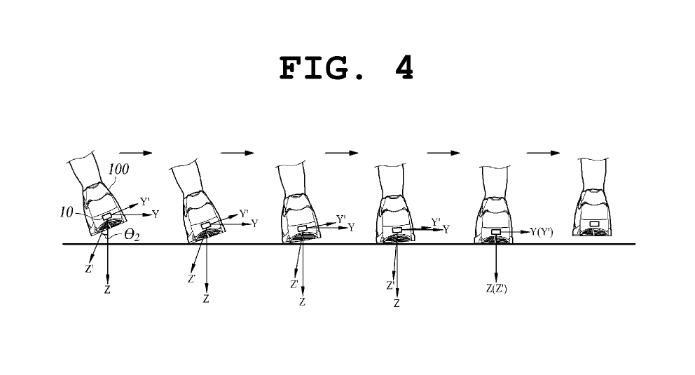

Such a sensor module 10 includes an accelerometer 11, a transmission unit 12, and a power supply unit 13. The accelerometer 11 may be one of a 1-axis accelerometer, a 2axis accelerometer, and a 3-axis accelerometer. When the accelerometer 11 is the 1-axis accelerometer, the rolling direction and the rolling angle of the shoe 100 with respect to an x axis which is the movement (walking) direction of the pedestrian are detected. That is, the accelerometer 11 detects whether the shoe 100 is accelerated in the inward direction of a body, as shown in FIG. 3, or whether the shoe 100 is accelerated in the outward direction of the body, as shown in FIG. 4, and remotely provides detected information through the transmission unit 12.

The transmission unit 12 is configured to transmit signals detected by the accelerometer 11 in a wireless manner, may be implemented using various types of wireless transmission modules, and may use various types of wireless transmission schemes without being limited to a single specific wireless communication scheme. The information transmitted from the transmission unit 12 is provided to the reception unit 21.

The power supply unit 13 is configured to supply electric energy required for the accelerometer 11 and the transmission unit 12, and may include, for example, a rechargeable lithium-ion battery.

The reception unit 21 may receive information provided by the transmission unit 12 and various types of information provided via a wireless network 40, and the received information may be transferred to the pattern analysis unit 23. The reception unit 21 may include an antenna installed in an electronic device carried by the pedestrian, for example, a mobile device including a smart phone.

That is, the system 20 for analyzing the walking pattern of the pedestrian according to the embodiment of the present invention may be provided through a mobile device such as a smart phone, or provided as a separate system.

The transmission unit 22 may transmit data via the wireless network 40 including a WiFi network, or a wired communication network.

The pattern analysis unit 23 analyzes the rolling information of the shoe 100 received through the reception unit 21, and determines a pattern (a footprint shape) touching the ground with respect to the movement direction x of the shoe 100.

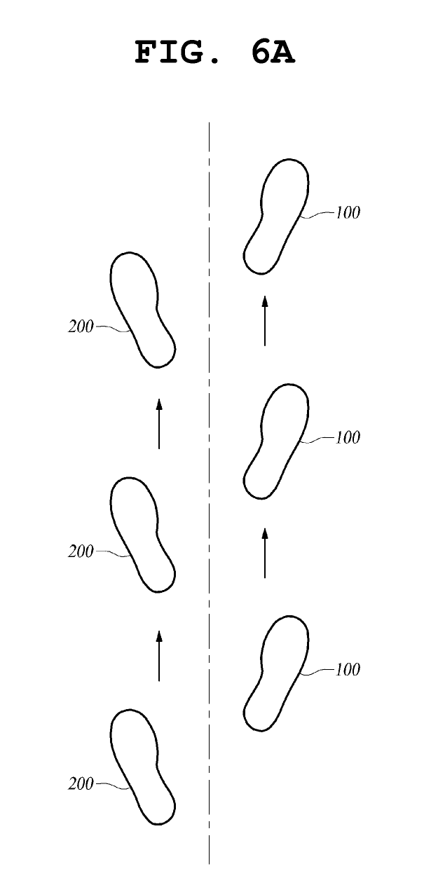

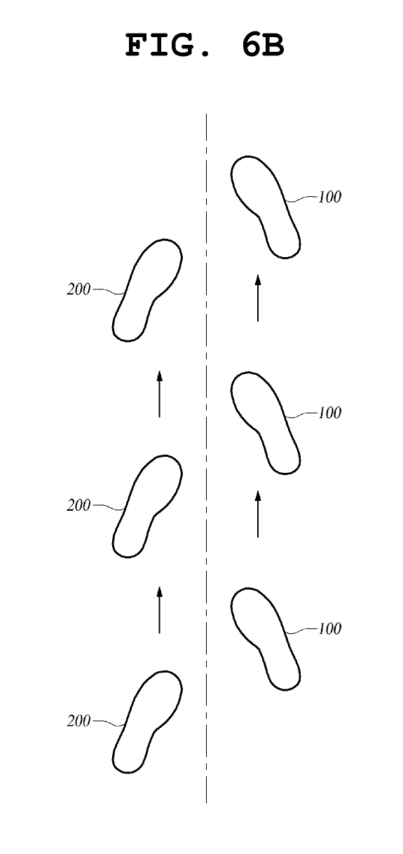

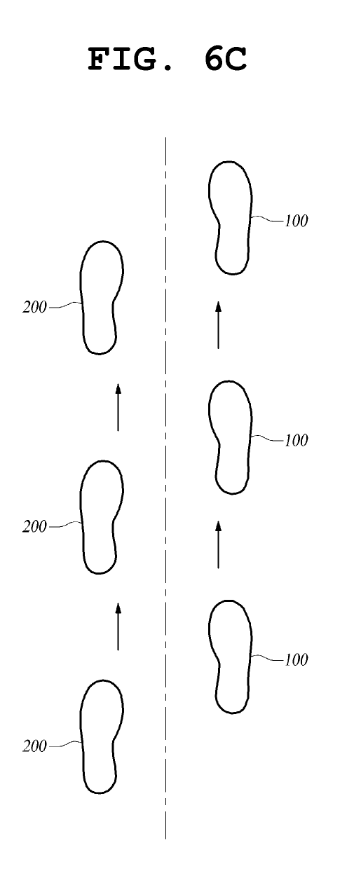

That is, as shown in FIG. 3, based on the right shoe 100, from a time point at which the heel of the shoe 100 initially touches the ground to a time point at which the outsole of the shoe 100 fully touches the ground, the sensor module 10 detects displacement coordinates (z′,y′) based on normal coordinates (z,y) via the accelerometer 11, and outputs the detected displacement coordinates through the transmission unit 12. Then, the pattern analysis unit 23 may calculate a rolling angle θ1=arctan(z′/y′) based on the output displacement coordinates (z′,y′), analyze the acceleration direction of the right shoe 100, and then determine that the pattern (footprint shape) obtained at a time point at which the right shoe 100 fully touches the ground is the shape shown in FIG. 6A. That is, the pattern analysis unit 23 may analyze the received detected data, calculate the rolling direction (acceleration direction) of the right shoe 100 as being the inward direction of the body, and then determine that the pattern (footprint shape) obtained at a time point at which the right shoe 100 and the left shoe 200 touch the ground is the shape shown in FIG. 6A.

The walking pattern determination unit 24 determines the walking pattern of the pedestrian based on the pattern of the shoe 100 touching the ground, determined by the pattern analysis unit 23. That is, when the pattern analysis unit 23 calculates the rolling direction (acceleration direction) of the shoe 100 as being the inward direction of the body, as shown in FIG. 3, the walking pattern determination unit 24 determines that the walking pattern of the pedestrian who wears the shoe 100 is that of a so-called out-toed gait.

Meanwhile, in the case of an in-toed gait that is contrary to the out-toed gait, that is, when a footprint pattern formed in the movement direction of the pedestrian is as shown in FIG. 6B, the walking pattern determination unit 24 determines that the walking pattern is that of the in-toed gait. In this case, the pattern analysis unit 23 may calculate a rolling angle θ2=arctan(z′/y′) based on the information detected by and transferred from the sensor module 10, that is, output displacement coordinates (z′,y′), analyze the acceleration direction of the right shoe 100, and then determine that the pattern (footprint shape) obtained at a time point at which the right shoe 100 fully touches the ground is the shape shown in FIG. 6B. That is, the pattern analysis unit 23 may analyze the received detected data, calculate the rolling direction (acceleration direction) of the right shoe 100 as being the outward direction of the body, and then determine that the pattern (footprint shape) obtained at a time point at which the right shoe 100 and the left shoe 200 touch the ground is the shape shown in FIG. 6B.

In this case, the walking pattern determination unit 24 determines that the walking pattern of the pedestrian is that of a so-called in-toed gait, and stores the determined information in the storage unit 25.

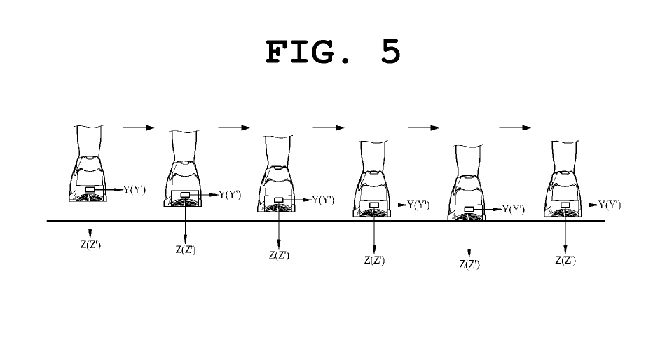

Meanwhile, FIG. 5 is a rear view showing states varying from a time point at which the heel of the right shoe 100 of the pedestrian initially touches the ground to a time point at which the entire outsole of the shoe 100 touches the ground, and illustrates a normal walking pattern, that is, a parallel-footed gait. In this case, the pattern analysis unit 23 may calculate a rolling angle θ=arctan(z′/y′) based on the information detected by the sensor module 10, that is, displacement coordinates (z′,y′), analyze the acceleration direction of the right shoe 100, and then determine that the pattern (footprint shape) obtained at a time point at which the right shoe 100 fully touches the ground is the shape shown in FIG. 6C. That is, the pattern analysis unit 23 may analyze the received detected data, calculate the rolling direction (acceleration direction) of the right shoe 100 as being neither the outward direction of the body nor the inward direction of the body, and then determine that the pattern (footprint shape) obtained at a time point at which the right shoe 100 and the left shoe 200 touch the ground is the shape shown in FIG. 6C.

Meanwhile, when the criteria for the determination of a normal walking pattern are determined as shown in FIG. 6C, a slight error may occur due to the shape of the ground on which the pedestrian walks or the like, and thus the determination of a normal walking pattern may be performed in such a way that, when the rolling direction occurs in the inward direction or the outward direction of the body within a preset allowable error range, the walking pattern can be determined to be a normal parallel-footed gait. For example, the range of the rolling angle θ=±5° is regarded as an allowable error range, and information detected within this allowable error range is ignored, and thus it can be determined that the walking pattern is a normal parallel-footed gait pattern. Such an allowable error may be collected and determined based on experimental data made through a plurality of examinees. Further, the allowable error may be preferably set in consideration of the physical characteristics of the pedestrian (weight, height, foot size, gender, age, etc.).

Therefore, a user, that is, the pedestrian, enters his or her physical characteristic information through the input unit 26, and then the input information is stored in the storage unit 25. Therefore, the walking pattern determination unit 24 may select an allowable error based on the input information of the pedestrian stored in the storage unit 25, and determine the walking pattern of the pedestrian. For this, the storage unit 25 may store reference values, that is, allowable errors or the like, based on the physical characteristics of the pedestrian in the form of a lookup table, and may provide the lookup table.

The above-described system for analyzing the walking pattern of the pedestrian according to the embodiment of the present invention may be provided through a portable electronic device, such as a smart phone, a mobile phone, or a portable computer, and is preferably provided by the service provision server 30 in the form of an application, thus allowing anyone who desires the application to use the system. The service provision server 30 includes a database (DB) 31, a support algorithm 32, and a transmission/reception unit 33. In the DB 31, pieces of data which are criteria for the determination of the walking pattern of each pedestrian are stored, and the pieces of data stored in the DB 31 may be continuously updated. Furthermore, pieces of information about users who are provided with the system of the present invention and use the system may also be stored and managed.

The support algorithm 32 is configured to provide an algorithm, that is, software, for analyzing the output data of the sensor module 10 installed on the shoe 100 and analyzing the walking pattern of the pedestrian, and may include an application provided when a request from the user is received via the wireless network 40 or the like.

The transmission/reception unit 33 is configured to exchange information with the reception unit 21 and the transmission unit 22 of the system 20 via the wireless network 40 or the wired network.

As described above, in accordance with the system 10 for analyzing the walking pattern of the pedestrian according to the embodiment of the present invention, detected data output from the single sensor module 10 installed on the shoe 100 is analyzed, and then it can be easily determined whether the walking pattern of the pedestrian is an in-toed gait, an out-toed gait, or a normal gait. Therefore, in order to determine the walking pattern of a pedestrian as in the case of the prior art, there is no need to install a plurality of sensors (modules) on a single shoe, thus not only reducing cost, but also facilitating the maintenance thereof.

In particular, even if the sensor module 10 is installed only on one shoe 100 of a pair of shoes 100 and 200, the walking pattern can be exactly analyzed, so that cost can be proportionally reduced, the time required to analyze the walking pattern can be reduced, and simplification can be realized. Therefore, the walking pattern of a pedestrian can be detected using minimum cost and a simple configuration.

Although the preferred embodiments of the present invention have been disclosed for illustrative purposes, those skilled in the art will appreciate that various changes and modifications are possible, without departing from the scope and spirit of the invention as disclosed in the accompanying claims.

(Description of the Reference Numerals)

| | |
|---|---|
| 10: sensor module | 20: system for analyzing the walking |
| | pattern of pedestrian |
| 21: reception unit | 22: transmission unit |
| 23: pattern analysis unit | 24: walking pattern |
| | determination unit |
| 25: storage unit | 26: input unit |
| 30: service provision server | 100, 200: shoe |

1. A system for analyzing a walking pattern of a pedestrian, comprising:
a sensor module installed on a moving object moving in a pattern corresponding to a movement trajectory of a foot of a pedestrian and configured to detect rolling information based on a movement direction of the pedestrian as an axis;
a reception unit configured to receive the rolling information detected by the sensor module;
a pattern analysis unit configured to analyze the rolling information of the moving object received through the reception unit and determine a rolling direction of the moving object after the moving object touches a ground; and
a walking pattern determination unit configured to determine a walking pattern of the pedestrian based on the rolling direction of the moving object after the moving object touches the ground, the rolling direction being determined by the pattern analysis unit,
wherein the walking pattern determination unit is configured to,
if the rolling direction of the moving object is analyzed as being an outward direction of the pedestrian by the pattern analysis unit, determine that the walking pattern is that of an in-toed gait,
if the rolling direction of the moving object is analyzed as being an inward direction of the pedestrian, determine that the walking pattern is that of an out-toed gait, and
if the rolling direction is analyzed as being not biased to the inward or outward direction of the pedestrian, determine that the walking pattern is that of a normal gait.

2. The system of claim 1, wherein the sensor module comprises a 1-axis accelerometer for detecting an acceleration direction from a time point at which the moving object initially touches the ground to a time point at which the moving object fully touches the ground.

3. The system of claim 1, wherein the sensor module comprises a 3-axis accelerometer for detecting an acceleration direction from a time point at which the moving object initially touches the ground to a time point at which the moving object fully touches the ground.
